(19) United States
(12) Patent Application Publication
(10) Pub. No.: US 2013/0324899 A1
(43) Pub. Date: Dec. 5, 2013

(54) BRACE WITH A SECURING DEVICE

(71) Applicant: Lance W. Lance, Lawrenceville, GA (US)

(72) Inventor: Lance W. Lance, Lawrenceville, GA (US)

(21) Appl. No.: 13/894,983

(22) Filed: May 15, 2013

Related U.S. Application Data
(60) Provisional application No. 61/654,303, filed on Jun. 1, 2012.

(51) Int. Cl. A61F 5/01

(52) U.S. Cl. CPC A61F 5/0125
USPC 602/26; 602/23

(57) ABSTRACT

A brace system worn by a user includes a brace, a belt, a strap, a securing mechanism and an adjusting mechanism. The brace is configured to provide support for a limb of the user. The belt is configured to be worn securely by the user above the brace. The strap depends downwardly from the belt and is configured to provide vertical support to the brace. The securing mechanism is configured to secure the strap to the brace. The adjusting mechanism is configured to adjust a length of the strap so that the strap maintains a desired supporting tension on the brace.

### CROSS-REFERENCE TO RELATED APPLICATION(S)

This application claims the benefit of U.S. Provisional Patent Application Ser. No. 61/654,303, filed Jun. 1, 2012, the entirety of which is hereby incorporated herein by reference.

### FIELD OF THE INVENTION

The present invention relates to medical devices and, more specifically, to a device for securing a knee brace and the like.

### DESCRIPTION OF THE RELATED ART

Knee braces are commonly used to support the knee of a wearer. A person who has undergone knee surgery or who has sustained a knee injury will wear a knee brace to prevent lateral movement of the lower leg relative to the thigh while still allowing forward flexing of the knee. Athletes will also wear knee braces to prevent knee injury that could result from lateral force being put on the knee, such as resulting from a side tackle in football.

Most knee braces include two rigid hinging elements that are secured to the opposite sides of the knee via an upper cuff and a lower cuff. The hinging elements must be aligned with the knee so that they allow proper flexure of the knee. The upper cuff is secured to the wearer's thigh, while the lower cuff is secured to the wearer's lower leg.

While the cuffs secure the brace to the knee, knee braces often slip down when in use. Once a brace slips, it is no longer in the position that allows proper knee flexure. As a result, the user must pull the brace back into the correct position. In the case of a knee healing from a knee injury or surgery, the cuffs may need to be released and reapplied to the user's leg to prevent further injury. This process can be irritating and time consuming.

Therefore, there is a need for a system that maintains a knee brace in its proper position.

### SUMMARY OF THE INVENTION

The disadvantages of the prior art are overcome by the present invention which, in one aspect, is a brace system worn by a user that includes a brace, a belt, a strap, a securing mechanism and an adjusting mechanism. The brace is configured to provide support for a limb of the user. The belt is configured to be worn securely by the user above the brace. The strap depends downwardly from the belt and is configured to provide vertical support to the brace. The securing mechanism is configured to secure the strap to the brace. The adjusting mechanism is configured to adjust a length of the strap so that the strap maintains a desired supporting tension on the brace.

In another aspect, the invention is a knee support system that includes a knee brace. A belt is configured to be worn about a user's waist. A strap depends downwardly from the belt and is configured to provide vertical support to the knee brace. A securing mechanism is configured to secure the strap to the knee brace. An adjusting mechanism is configured to adjust a length of the strap so as to maintain a desired supporting tension on the brace.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is an elevational view of one embodiment of a device for securing a brace.

FIGS. 2A and 2B are elevational views of the device shown in FIG. 1 applied to a knee brace.

FIG. 3 is an elevational view of an alternate embodiment of a device for securing a knee brace that includes more than one supporting strap.

FIG. 4 is an elevational view of an alternate embodiment of a device for securing a knee brace that includes a wrap-around supporting strap.

FIGS. 5A and 5B are photographs of one representative embodiment of a device for securing a knee brace.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

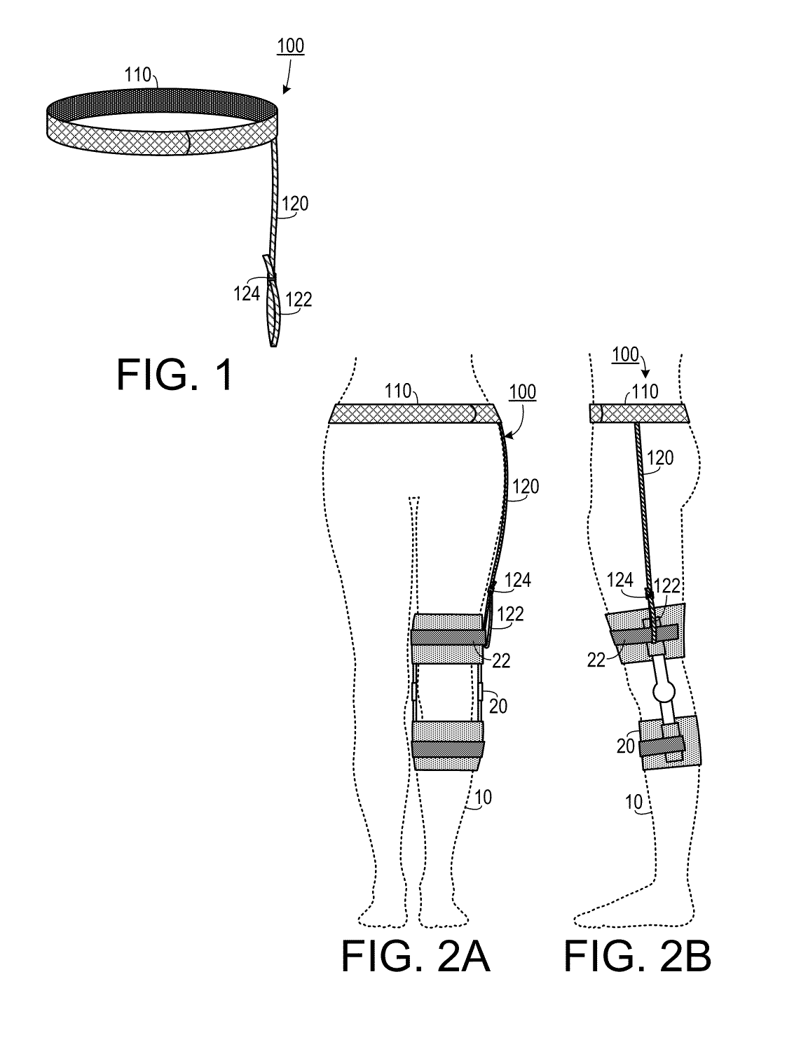

As shown in FIGS. 1, 2A, and 2B, one embodiment of a device 100 for securing a brace, such as a knee brace 20, includes a belt 110 with an elongated strap 120 depending downwardly therefrom. The belt 110 would typically be made from a material that can comfortably provide vertical support to the strap 120, including reinforced nylon, leather, etc. The strap 120 could be made from one of many materials including nylon webbing, ribbon, leather, etc. The strap 120 can be secured to the belt 110 using one of many methods, including: looping a top portion of the strap 120 about the belt; temporarily affixing the strap 120 to the belt 110 (e.g., with a hook-and-loop fastener); and affixing the strap 120 permanently to the belt 110 (e.g., by sewing or riveting, etc.).

The strap 120 includes a device for securing it to the brace 20. For example, the strap 120 can include a bottom loop 122 that can be looped around a securing portion 22 of the upper cuff of the brace 20. The belt 110 is worn about the waist of the user 10 and the strap 120 provides vertical support to the brace 20. A moveable clasp 124 can be used to adjust the length of the strap 120, thereby ensuring that the correct supporting tension is provided to the brace 20. (As one of skill in the art would readily recognize, a similar clasp can be employed at the top of the strap 120 for adjusting its length.)

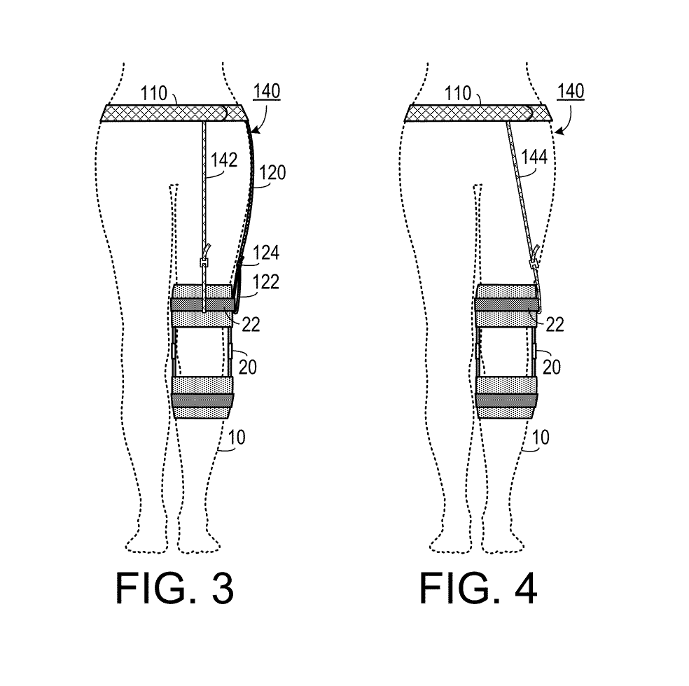

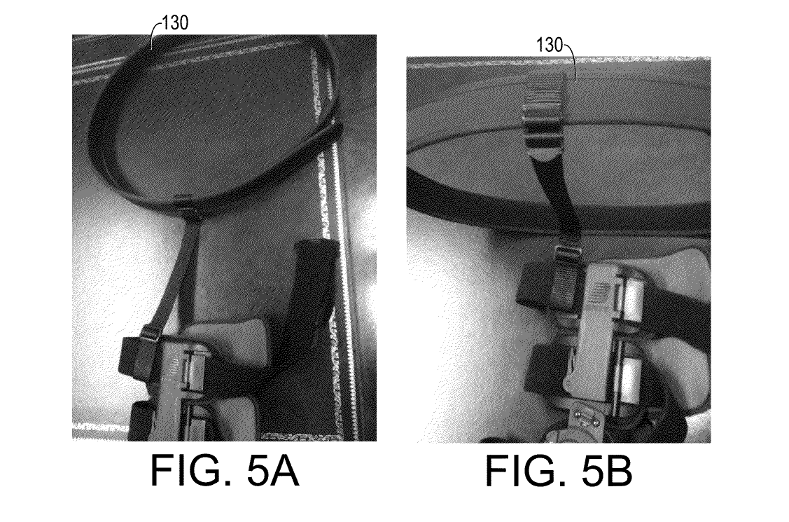

As shown in FIG. 3, an alternate embodiment of a brace securing device 140 can employ more than just a single side strap 120 to support the brace 20. For example, it can employ and additional front strap 142 or an additional back strap (not shown), or both. Additional straps may be useful if the brace 20 is subject to a high level of activity. As shown in FIG. 4, the strap 144 can be anchored to either the front or the back of the belt 110 and can wrap around to the side of the brace 20. A prototype embodiment 130 of a device for securing a knee brace is shown in FIGS. 5A and 5B.

Also, a brace supporting device can be used to support other types of braces. For example, one embodiment (not shown) could be used to support an elbow brace. Such a device would include a belt or other support that is securable to the wearer's shoulder and a strap that couples the support to the brace.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A brace system, comprising:
(a) a brace configured to provide support for a limb of a user;
(b) a belt configured to be worn securely by the user above the brace;
(c) a strap depending downwardly from the belt and configured to provide vertical support to the brace;
(d) a securing mechanism configured to secure the strap to the brace; and
(e) an adjusting mechanism configured to adjust a length of the strap so that the strap maintains a desired supporting tension on the brace.

2. The device of claim 1, wherein the brace comprises a knee brace.

3. The device of claim 2, wherein the belt is configured to be worn about a waist of the user.

4. The device of claim 1, wherein the belt comprises a reinforced nylon belt.

5. The device of claim 1, wherein the strap comprises a nylon web strap.

6. The device of claim 1, wherein the strap comprises a side strap and further comprising a front strap spaced apart from the side strap and depending downwardly from the belt and configured to provide vertical support to the brace.

7. The device of claim 1, wherein the adjusting mechanism comprises a slideable clasp.

8. A knee support system, comprising:
(a) a knee brace;
(b) a belt configured to be worn about a user's waist;
(c) a strap depending downwardly from the belt and configured to provide vertical support to the knee brace;
(d) a securing mechanism configured to secure the strap to the knee brace; and
(e) an adjusting mechanism configured to adjust a length of the strap so as to maintain a desired supporting tension on the brace.

9. The knee support system of claim 8, wherein the belt comprises a reinforced nylon belt.

10. The knee support system of claim 8, wherein the strap comprises a nylon web strap.

11. The knee support system of claim 8, wherein the strap comprises a side strap and further comprising a front strap spaced apart from the side strap and depending downwardly from the belt and configured to provide vertical support to the brace.

12. The knee support system of claim 8, wherein the adjusting mechanism comprises a slideable clasp.
